(19) United States
(12) Patent Application Publication
(10) Pub. No.: US 2026/0137601 A1
(43) Pub. Date: May 21, 2026

(54) INTEGRATED BIOMIMETIC ORAL-TACTILE DEVELOPMENT SYSTEM FOR EARLY CHILDHOOD SENSORY TRAINING

(71) Applicant: Scott Henderson Design LLC, Brooklyn, NY (US)

(72) Inventor: Scott Henderson, Brooklyn, NY (US)

(73) Assignee: Scott Henderson Design LLC, Brooklyn, NY (US)

(21) Appl. No.: 19/452,505

(22) Filed: Jan. 19, 2026

Related U.S. Application Data
(63) Continuation-in-part of application No. 63/824,373, filed on Jun. 16, 2025.
(63) Continuation-in-part of application No. 29/972,052, filed on Nov. 7, 2024.

(51) Int. Cl. A61J 17/00; A61M 21/02; A61M 21/00

(52) U.S. Cl. CPC A61J 17/109; A61J 17/001; A61M 21/02; A61M 2021/0022; A61M 2021/0027; A61M 2021/005; A61M 2205/0216; A61M 2205/59

(57) ABSTRACT

The present invention relates to a biomimetic sensory engagement system configured to promote oral, tactile, and visual development in infants and young children. The system includes a unitary body formed of food-grade silicone having a hollow interior that defines a compressible volume, a pacifier-shaped oral-engagement element, a prehensile extension adapted to wrap around a support, and textured exterior regions that stimulate fine-motor and sensory exploration. In certain embodiments, the body incorporates wall-thickness mapping to provide differentiated firmness zones, and a handle loop integrally formed with the body to support early grasping skills. The spatial arrangement of the oral and prehensile regions defines a recognizable biomimetic form, such as a seahorse, monkey, or dolphin, encouraging child recognition and voluntary interaction. The invention provides a safe, monolithic structure that enhances developmental engagement, hygiene, and ease of handling compared with conventional multi-piece teethers or toys.

### CROSS-REFERENCE TO RELATED APPLICATIONS

This application contains subject matter that is related to the subject matter of the following co-pending application. The below-listed application is hereby incorporated herein by reference in its entirety:

This is a U.S. non-provisional application that is a continuation in part of a U.S. non-provisional application, Serial No. 63/824,373, inventor Scott Henderson, entitled “MULTI-SENSORY TEETHING TOY WITH INTEGRATED GRIPPING, SOOTHING, AND ATTACHMENT FEATURES”, filed June 16, 2025; and

a continuation in part of a U.S. design application, Serial No. 29/972,052, inventor Scott Henderson, entitled “TEETHER”, filed November 7, 2024.

### TECHNICAL FIELD OF THE INVENTION

This invention relates to sensory-development devices for infants and young children, and particularly to biomimetic systems configured to promote oral, tactile, and visual engagement through a unified, monolithic structure. The invention further relates to infant oral-comfort and grasp-training apparatuses formed from food-grade silicone materials, incorporating pacifier-shaped oral elements, prehensile extensions, and textural surface regions that encourage multi-sensory interaction and fine-motor development. The invention also pertains to infant sensory systems having wall-thickness mapping or differentiated firmness zones that deliver enhanced feedback during chewing, grasping, and compression, while maintaining hygienic, one-piece construction suitable for repeated cleaning and safe use under adult supervision.

### BACKGROUND OF THE INVENTION

Before our invention, teething toys and baby sensory aids were commonly designed with limited scope and functionality, often falling short of addressing the full spectrum of baby developmental needs and caregiver practicalities. Prior approaches generally focused on providing a simple surface for chewing, neglecting opportunities to engage multiple sensory pathways that are critical during early-stage cognitive and motor development. While chewing relief was often the primary goal, the lack of tactile variety and auditory or visual cues meant that babies quickly lost interest, limiting the utility and lifespan of such products.

Many prior teething devices were constructed using a combination of different materials—such as hard plastics, gels, or embedded components—that introduced safety concerns. These multi-material assemblies were more prone to wear, failure at seams or joints, and incompatibility with common sterilization and cleaning methods. Over time, components could degrade, detach, or become contaminated, posing risks that made extended use impractical or unsafe. In some cases, external coatings or painted details were used to enhance visual appeal, but these finishes were subject to degradation and could introduce additional ingestion hazards.

Attachment and portability were also frequently overlooked. Some designs required third-party accessories like clips, straps, or separate fasteners to secure the toy during travel or when not in use. These extra components added bulk, introduced additional potential failure points, and complicated the caregiver’s experience. Such accessories also created points of entanglement or entrapment, especially in the context of crib or stroller use. Without an integrated means of attachment, many teethers were easily dropped or lost, resulting in increased replacement costs and diminished hygiene.

Ergonomics presented another challenge in prior designs. Handle structures, if present, were often awkwardly sized or shaped for developing baby hands. Some products lacked gripping zones entirely, forcing the baby to manipulate the toy by its body or chewing surfaces, which increased the likelihood of dropping and made intentional use difficult. This limitation reduced opportunities for building hand-eye coordination and motor planning skills.

Finally, many existing teething toys failed to support emotional or visual engagement. While bright colors or animal shapes were sometimes included to draw attention, they often served as superficial design elements rather than calming or developmentally supportive features. Few prior designs recognized the importance of visual calmness and emotional reassurance, particularly during moments of teething discomfort when overstimulation can worsen a baby’s distress.

The present invention addresses these and other shortcomings by providing a biomimetic sensory engagement system that unifies teething relief, tactile stimulation, and emotional comfort within a single, seamless structure inspired by recognizable animal forms. By organizing oral, visual, and grasping features into a cohesive topology that evokes natural anatomy, the invention promotes intuitive interaction by the child while ensuring safety, hygiene, and manufacturing simplicity. For these reasons and shortcomings, as well as other reasons and shortcomings, there exists a long-felt need for a biomimetic, monolithic system that fosters oral-motor development, sensory exploration, and emotional soothing in a single integrated design.

### SUMMARY OF THE INVENTION

The shortcomings of the prior art are overcome, and additional advantages are provided through the provision of a biomimetic sensory engagement system configured to promote oral, tactile, and visual interaction by a child. The system includes a unitary body formed of food-grade silicone defining a hollow interior that compresses to generate responsive tactile feedback. A pacifier-shaped element projects from a first region of the body to form an oral-engagement zone that safely accommodates teething and mouthing activity. A prehensile extension extends from the body and is configured to wrap around a support structure and retain its shape through the elastic memory of the silicone material. One or more tactile-texture zones are positioned along exterior surfaces to enhance sensory exploration and fine-motor control. The oral and prehensile regions are spatially arranged along a longitudinal axis to create a recognizable biomimetic form, such as an animal, encouraging voluntary engagement and developmental interaction by the child.

Additional shortcomings of the prior art are overcome, and additional advantages are provided through the provision of a biomimetic sensory engagement system configured to promote oral, tactile, and visual interaction by a child. The system includes a unitary body formed of food-grade silicone having a hollow interior that compresses to deliver tactile feedback during handling. A pacifier-shaped element projects from a first region of the body to define an oral-engagement zone formed by a region of increased wall thickness relative to adjacent portions of the body. A prehensile extension is configured to wrap around a support structure and maintain its shape through the elastic memory of the silicone material. At least one tactile-texture zone is distributed on the exterior surface to stimulate fine-motor and sensory response. The body further includes a wall-thickness mapping that defines multiple zones of firmness, allowing the child to experience progressive resistance and enhanced sensory engagement across different regions of the biomimetic form.

Additional shortcomings of the prior art are overcome, and additional advantages are provided through the provision of a biomimetic sensory engagement system configured to promote oral, tactile, and visual interaction by a child. The system comprises a monolithic body formed of food-grade silicone that integrally defines multiple functional regions within a single, seamless structure. A pacifier-shaped oral-engagement portion includes a localized region of increased wall thickness forming a chew zone that safely accommodates mouthing and teething behavior. A hollow torso portion provides a compressible cavity that generates responsive tactile feedback when squeezed, while a prehensile tail is configured to elastically wrap around a support for convenient hanging or manipulation. The oral and tail regions are spatially arranged to emulate the anatomical head and tail of an animal, thereby defining a recognizable biomimetic topology that encourages visual recognition, voluntary grasping, and multi-sensory engagement by the child.

Additional features and advantages are realized through the techniques of the present invention. Other embodiments and aspects of the invention are described in detail herein and are considered a part of the claimed invention. For a better understanding of the invention with advantages and features, refer to the description and the drawings.

### BRIEF DESCRIPTION OF THE FIGURES

The subject matter, which is regarded as the invention, is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates one example of a side view of a multi-sensory biomimetic sensory engagement system;

FIG. 2 illustrates one example of a cross-sectional side view of a multi-sensory biomimetic sensory engagement system;

FIGS. 3-6 illustrates examples of perspective views of a multi-sensory biomimetic sensory engagement system;

FIG. 7 illustrates one example of a front view of a multi-sensory biomimetic sensory engagement system;

FIG. 8 illustrates one example of a back view of a multi-sensory biomimetic sensory engagement system;

FIG. 9 illustrates one example of a top view of a multi-sensory biomimetic sensory engagement system;

FIG. 10 illustrates one example of a bottom view of a multi-sensory biomimetic sensory engagement system; and

FIG. 11 illustrates examples of recognizable biomimetic topologies.

The detailed description explains the preferred embodiments of the invention, together with advantages and features, by way of example, with reference to the drawings.

### DETAILED DESCRIPTION OF THE INVENTION

Teething is a developmental milestone in infancy characterized by discomfort, irritability, and a natural drive for oral stimulation as primary teeth begin to emerge. During this period, babies instinctively seek out objects they can chew on to alleviate gum pain and pressure. In response to this need, the market offers a wide array of teething aids and baby toys. However, most conventional solutions fall short of addressing the full range of physical, sensory, and developmental requirements associated with teething. Existing teething toys frequently emphasize a singular function—such as chewing relief—while neglecting important complementary features such as safe graspability, hygienic storage, multi-sensory stimulation, and secure attachment for on-the-go use. Others may attempt to offer more interactivity through added components or hybrid materials, but these designs often increase manufacturing complexity, raise cost, introduce potential choking hazards, or compromise durability and safety.

Furthermore, many teething products rely on separate pieces or detachable attachments—such as pacifier clips, handles, or hard inserts—that require additional assembly or may degrade over time. Inconsistent material choices within a single product can also present problems, including poor washability, chemical incompatibility, or failure to meet stringent safety regulations for baby use. A critical need exists for a teething solution that is fully integrated, monomaterial, and capable of delivering a broad range of sensory interactions while preserving safety, manufacturability, and durability.

The present invention addresses these challenges by providing a multi-sensory biomimetic sensory engagement system that is formed entirely from food-grade silicone using a unified molding process. All functional and interactive components of the toy are designed into a seamless body with localized material properties achieved through strategic variations in wall thickness. Rather than assembling separate parts, this construction method produces a toy that appears and behaves as a single cohesive unit, enhancing structural integrity while simplifying cleaning and reducing production complexity.

In an exemplary embodiment, the multi-sensory biomimetic sensory engagement system is formed entirely from 100% food-grade silicone, offering substantial advantages over other materials used in similarly sized baby toys. Unlike natural rubber, which fades in sunlight, emits a strong odor, and may melt or deform under heat exposure, food-grade silicone is color-stable, heat-resistant, odorless, hypoallergenic, and safe for oral use. This material remains dimensionally stable even in high-heat environments such as sunny windowsills or near dish drying areas. In contrast to natural rubber, which can trigger allergic reactions and carries a taste, the present material is chemically neutral and safe for extended oral engagement.

In addition to its performance characteristics, the material has sustainability and ethical sourcing benefits. Food-grade silicone is derived primarily from silica (sand), one of the most abundant natural resources on Earth. The material is also recyclable, particularly when kept pure and not bonded with other materials, as in the current toy. By contrast, natural rubber is harvested from Hevea trees in limited geographic zones such as tropical rainforests, and its extraction processes are often associated with deforestation and unregulated labor conditions. The present invention avoids these environmental and ethical issues entirely, providing a high-quality, single-material solution that is both safer for children and more responsible for the planet.

Although the multi-sensory biomimetic sensory engagement system is constructed from heat-resistant, food-grade silicone and could withstand placement on the top rack of a dishwasher without deformation or color fading, the recommended care method is hand washing only. This guideline is intended to preserve surface integrity and avoid exposure to harsh dishwasher detergents, which may be a concern for some caregivers. The multi-sensory biomimetic sensory engagement system’s geometry—including the hollow body, integrated handle, and encapsulated squeaker—further supports rapid drying when suspended by its prehensile tail. Sterilization through boiling or chemical solutions is not recommended, and the multi-sensory biomimetic sensory engagement system packaging explicitly instructs hand-wash-only cleaning.

Key features of the invention include a compressible hollow body that allows for deformation and houses a securely encapsulated squeaker, a spiral tail configured as a prehensile structure capable of wrapping around external supports, and a loop-shaped handle designed for baby grip development. Oral engagement is enhanced through specific zones of increased rigidity—such as gum massagers formed as pronged mane structures and chew regions placed along the front and back torso—that provide firm, yet safe, surfaces for gnawing. These elements are purposefully reinforced through localized thickening of the silicone walls without the need for additional materials or inserts.

In an exemplary embodiment, the structural and functional features of the invention are adapted to various animal-themed characters, such as seahorses, monkeys, dolphins, and other creatures, each offering unique opportunities for incorporating the prehensile and oral engagement elements. For example, in a seahorse configuration, the curled tail naturally lends itself to a spiral or hook-like structure suitable for wrapping around stroller bars or crib rails. Similarly, a monkey-themed variant may feature an extended tail that doubles as a flexible hanging appendage while incorporating gum-massaging surface textures along its limbs or torso. In a dolphin character, the prehensile tail can become a dorsal hook, allowing for intuitive hanging while preserving the chewable, squeezable, and squeaking features consistent with the core invention. These animal forms not only reinforce the functional elements of the toy but also enhance visual and thematic appeal, making them emotionally engaging and developmentally beneficial for babies.

In certain embodiments, the invention incorporates a closed handle structure that is both functionally ergonomic and visually integrated into the overall character design. The handle takes the form of a fully enclosed loop molded from the same food-grade silicone material as the rest of the toy, offering a secure grip for young children while maintaining structural softness and pliability. Unlike traditional teething toys that feature prominent external rings or add-on through-holes for handling, the present invention conceals the handle within the character’s silhouette—for example, seamlessly blending it into the body arc of a stylized seahorse. As a result, the toy preserves its aesthetic integrity while providing a graspable structure that supports early-stage grip development and reduces the likelihood of accidental drops. The closed handle is not an obvious ring but an integrated contour within the toy’s geometry, creating a uniquely camouflaged gripping feature that is both innovative and visually cohesive. This design discourages direct mimicry by competitors while offering practical usability for the child and aesthetic delight for caregivers.

In addition to facilitating attachment to stroller bars, crib rails, and diaper bags during on-the-go use, the prehensile tail structure also enables convenient drying of the toy following hand washing. Parents may use the tail to hang the toy from kitchen cabinet knobs, drying racks, or towel rails, thereby promoting hygienic storage without the need for direct contact with flat or unsanitary surfaces. This dual-use functionality enhances the utility of the tail design, making it suitable not only for transport but also for post-cleaning convenience in the home environment.

To further support cognitive and emotional development, the toy includes visual elements such as a molded sleeping-eye indicator that imparts a calming aesthetic and reinforces the toy’s soothing role. Distributed texture zones across the exterior of the toy promote tactile exploration and motor coordination by introducing subtle variations in surface geometry. The flat, widened base allows the toy to rest upright on flat surfaces, preserving hygiene by elevating the oral contact regions above dirty surfaces and enhancing visual accessibility to the child.

By integrating these features into a single monolithic structure, the present invention achieves a unique combination of functional performance, user safety, sensory enrichment, and developmental support. It eliminates the drawbacks of multi-piece designs and introduces a higher level of manufacturing efficiency. The toy's ability to be wrapped, gripped, chewed, squeezed, and explored—without requiring any modification or assembly—makes it particularly well suited for both at-home and on-the-go use by babies under adult supervision. In addition, the simplicity of the construction of the biomimetic sensory engagement system 100, a toy for use by a child, is compliant with regulatory safety standards, such as ASTM F963, and provides a strong barrier to commoditized replication, due in part to the sophisticated molding techniques involved in manufacturing its hollow, multi-thickness form.

In this regard, in an exemplary embodiment, the unitary body 102 can be monolithic and entirely free of seams, joints, or detachable parts. The food-grade silicone composition permits hand-washing or dishwasher cycles without deformation or color change. This single-material construction ensures long-term durability and compliance with safety regulations, such as ASTM F963 and CPSIA standards.

In further embodiments, the present invention extends beyond a traditional teething aid to form an integrated biomimetic sensory engagement system that holistically supports the child’s oral, tactile, and visual development. The system is defined by a unitary body molded entirely from food-grade silicone, in which distinct interactive zones—including oral-engagement regions, prehensile extensions, and tactile-texture surfaces—are integrated into a single, continuous form. This structural integration allows the device to function as a dynamic developmental platform rather than a static object, facilitating responsive sensory feedback through elastic deformation, variable firmness, and multi-directional handling.

A key advancement of the present invention is the implementation of wall-thickness mapping, wherein local variations in the silicone wall geometry establish multiple zones of tactile firmness throughout the body. Regions intended for chewing or oral engagement are formed with increased thickness and density, while adjacent hollow regions remain thinner and more compressible. This contrast enables a graduated sensory response that evolves with applied force, providing an engaging, self-exploratory experience for infants developing oral–motor coordination. The resulting tactile pattern simulates the natural variability found in biological forms, reinforcing the biomimetic theme while offering developmental benefit through differentiated feedback.

In certain embodiments, a pacifier-shaped element protrudes from a first region of the body to define a dedicated oral-engagement zone. This portion may include smooth, contoured lips or pronged ridges to massage gum tissue during mouthing. The opposite end of the body incorporates a prehensile extension, such as a spiral or tail-like structure, that can wrap around a support (e.g., stroller handle, crib rail, or caregiver finger) and retain its wrapped configuration through the elastic memory of the silicone material. This prehensile capability allows for hands-free storage and hygienic drying after washing, reducing caregiver handling and promoting sanitary use in diverse environments.

Beyond its mechanical design, the invention leverages visual and emotional cues to enhance developmental engagement. In certain embodiments, the unitary body incorporates a molded or recessed visual indicator, such as a sleeping-eye design, which functions as a calming symbol recognizable by both child and caregiver. The sleeping-eye indicator contributes to emotional conditioning—associating the product with soothing routines such as rest or comfort—and supports visual focus and recognition in infants developing early-stage perception.

Additional embodiments may feature distributed tactile textures across the body surface, such as ridges, dimples, or nubs that vary in height, pattern, and spacing. These micro-textures promote sensory exploration and fine-motor development by stimulating nerve endings in the lips, tongue, and fingertips. The tactile elements may also be strategically positioned to align with animal-like features, reinforcing the biomimetic geometry of the system.

The system’s overall spatial arrangement—with oral-engagement and prehensile regions aligned along a longitudinal axis—creates a recognizable animal-inspired silhouette, such as a seahorse, monkey, dolphin, or similar creature. This biomimetic form serves dual purposes: it visually attracts the child through familiarity and animacy while structurally distributing interactive regions in a balanced, ergonomic layout. The recognizable shape encourages voluntary engagement, allowing infants to initiate self-directed exploration based on instinctive attraction to living forms.

In certain embodiments, the invention also includes a closed handle loop integrated seamlessly within the silhouette of the body. The handle loop may define an enclosed gripping region accessible to small hands, promoting early grasp development and bilateral coordination. Unlike add-on handles that disrupt symmetry or require secondary attachment, this integrated handle structure is formed monolithically within the system’s geometry and preserves its overall aesthetic and hygienic continuity.

The biomimetic sensory engagement system may further include acoustic or airflow chambers incorporated within the hollow cavity, configured to emit a subtle squeak or airflow sound during compression. This optional acoustic response introduces an auditory dimension to the sensory experience, reinforcing cause-and-effect awareness and rewarding tactile exploration with sound feedback.

Altogether, these features transform a conventional teething object into a multi-sensory developmental platform that supports the child’s physical comfort, emotional regulation, and cognitive growth. By combining oral-engagement surfaces, compressible tactile zones, visual cues, and self-supporting prehensile structures within a single silicone form, the present invention achieves a rare integration of safety, functionality, and emotional resonance.

Accordingly, the present invention offers a comprehensive, thoughtfully engineered solution to the long-standing limitations of conventional teething aids. It provides a platform for oral soothing, sensory stimulation, emotional comfort, and fine motor skill development in a compact, durable, and hygienic form that is both practical for caregivers and deeply engaging for babies.

In the present invention, the term “biomimetic sensory engagement system” is intended to mean a child-safe, monolithic, silicone-based toy structure engineered to deliver combined oral, tactile, and visual sensory stimuli through coordinated geometry, texture, and material elasticity within a single molded form. The system is specifically configured for infant and toddler interaction, promoting developmental engagement, safe oral exploration, and caregiver convenience through its unified and hygienic construction.

In the present invention, the term "unitary body" is intended to mean a structure formed from a single, continuous molding process without seams, joints, or separable components, such that all included features are integrated into one monolithic form.

In the present invention, the term “prehensile extension” (also referred to as a “prehensile feature”) is intended to mean an elongated, tail-like structure integrally formed with the unitary body of the toy, configured to wrap around or secure itself to external supports such as bars, loops, or handles. The prehensile extension may take the form of a tail, hook, spiral limb, or other geometrically curved appendage and is characterized by the ability to retain its wrapped or coiled configuration through the elastic memory of the material, thereby enabling self-supporting attachment without external clips, straps, or fasteners.

In the present invention, the term "hollow body" is intended to mean a cavity-containing region within the toy that allows for deformation, compression, or sound generation, and which contributes to the toy’s pliability and interactive responsiveness.

In the present invention, the term "rigid body" is intended to mean a region within the toy that is made more structurally stiff through localized thickening of the wall material, providing greater resistance to deformation than adjacent areas of the hollow body.

In the present invention, the term "localized chew zone" is intended to mean an area of the toy configured for oral engagement, formed by selectively increasing wall thickness to create a firm, chewable surface that contrasts in tactile firmness from surrounding regions.

In the present invention, the term "gum massager" is intended to mean one or more protruding elements, such as prongs or nubs, formed integrally with the toy and configured to apply pressure to a baby’s gums for soothing purposes during chewing.

In the present invention, the term “handle loop” (also referred to as an “integrated handle”) is intended to mean a closed or partially closed gripping structure formed integrally with the unitary body of the toy. The handle loop maintains continuous material continuity with surrounding regions and is dimensioned for infant grasping from multiple orientations to promote fine motor development, hand-eye coordination, and secure handling. The integrated handle may take the form of a ring, arc, or enclosed contour that enables the child to lift, hold, or manipulate the toy without detachment or discontinuity in the molded body.

In the present invention, the term "visual indicator" is intended to mean a molded visual feature, such as recessed eyes or facial cues, configured to promote emotional calmness or suggest a resting state to support soothing behavior.

In the present invention, the term “tactile zone” (encompassing both “tactile texture zones” and “tactile firmness zones”) is intended to mean any surface or sub-surface region of the unitary body that provides distinct sensory feedback through either geometric texture or localized rigidity. A tactile texture zone refers to a surface area having molded-in texture, recesses, or raised patterns that differ from adjacent surfaces to stimulate the sense of touch and encourage exploratory handling. A tactile firmness zone refers to a region defined by controlled wall-thickness mapping that produces perceptible variations in softness or rigidity, generating contrasting oral or manual feedback. Together, these tactile zones create a coordinated sensory landscape that supports developmental engagement and multi-modal stimulation during child interaction.

In the present invention, the term "squeaker element" is intended to mean an internally enclosed acoustic device that emits sound upon compression of the toy’s body, and which is permanently sealed to prevent access or removal.

In the present invention, the term “wall-thickness mapping” (which may include a “localized firmness gradient”) is intended to mean a controlled distribution of material thicknesses within the molded body that establishes multiple zones of tactile firmness. Thicker regions define stiffer or more rigid sections suitable for oral engagement or structural attachment, while thinner regions remain compressible to enhance tactile responsiveness. In certain embodiments, the wall-thickness mapping transitions gradually between regions to form a localized firmness gradient, thereby providing progressive changes in material stiffness that generate differentiated oral or manual feedback aligned with varying developmental skill levels and pressure thresholds. The wall-thickness mapping is achieved within a single molding cycle without the use of inserts, joints, or secondary materials.

In the present invention, the term "sleeping-eye indicator" is intended to mean a visual indicator molded, recessed, or raised upon the outer surface of the toy that represents a closed or resting eye. The indicator is configured to convey a calming emotional state recognizable to both infants and caregivers, supporting soothing and rest-time association while also functioning as a tactile depression that can be traced by the child’s fingers.

In the present invention, the term "biomimetic topology" is intended to mean a spatial configuration or overall body form that emulates recognizable anatomical features of an animal—such as a head, torso, tail, or fins—thereby eliciting visual familiarity and instinctive engagement by the child.

In the present invention, the term "compressible cavity" is intended to mean an internal air or fluid-filled region of the body that deforms under applied pressure and returns to its original volume upon release, thereby generating responsive tactile and auditory feedback during use.

In the present invention, the term "elastic memory" is intended to mean the material’s inherent ability to return to a pre-formed shape after deformation, such that structures like the prehensile feature can repeatedly coil and uncoil without permanent distortion.

In the present invention, the term "stabilizing base" is intended to mean a widened or flattened region of the toy configured to support upright orientation when placed on a flat surface, thereby maintaining hygiene by preventing oral-contact portions from touching contaminated areas.

In the present invention, the term "acoustic channel" is intended to mean one or more internal passageways connecting the compressible cavity to a squeaker or resonant chamber, through which air flow during compression produces a controlled audible sound within infant-safe decibel limits.

In the present invention, the term "food-grade silicone" is intended to mean a medical- and food-safe elastomer derived primarily from silica, free of BPA, latex, and phthalates, capable of withstanding repeated washing and moderate heat exposure without degradation or loss of elasticity.

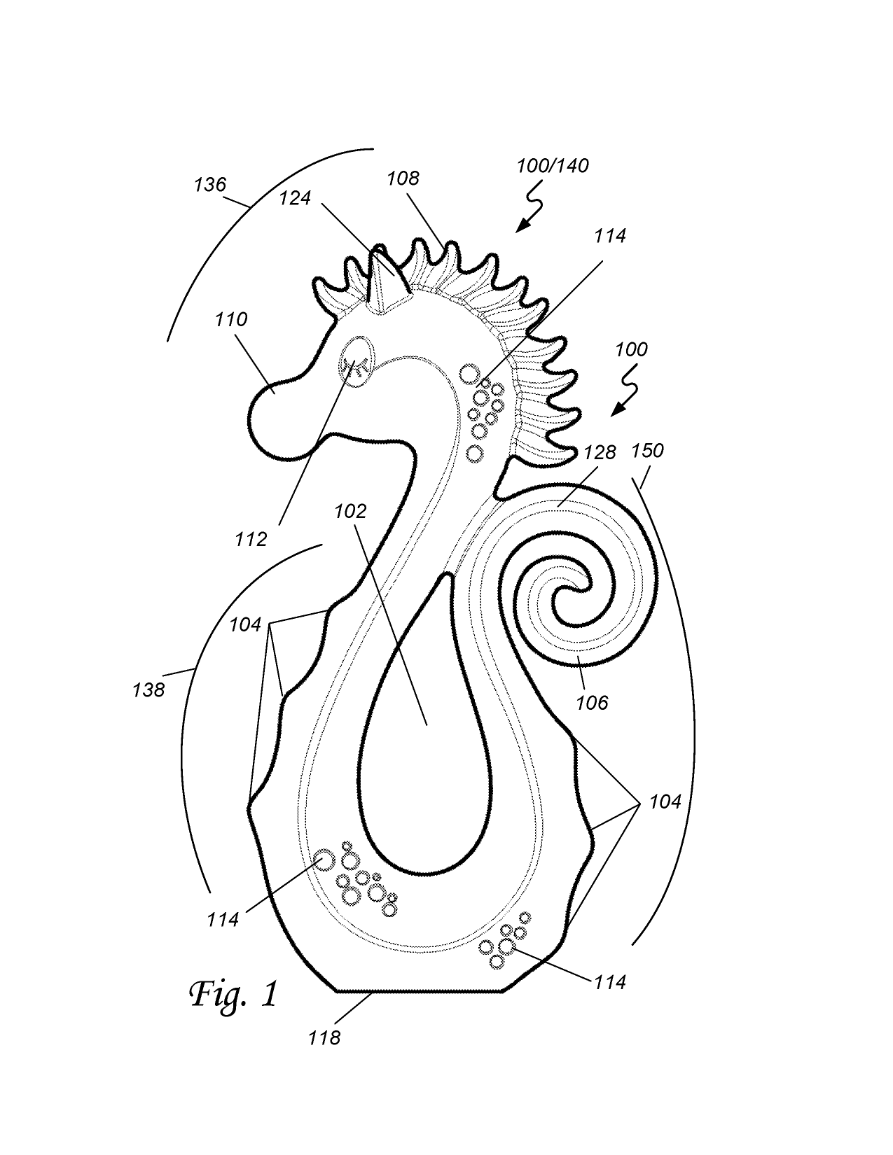

Turning now to the drawings in greater detail, it will be seen that in FIG. 1, there is illustrated one example of a side view of a biomimetic sensory engagement system 100. In an exemplary embodiment, the biomimetic sensory engagement system 100 is formed as a unitary, molded structure from food-grade silicone, incorporating a variety of features configured to enhance sensory development, self-soothing, and safe baby interaction. As the left and right sides of the toy are generally symmetrical, the side view illustrated in FIG. 1 is representative of both lateral aspects of the biomimetic sensory engagement system 100.

The biomimetic sensory engagement system 100 includes a unitary body 102 molded entirely from food-grade silicone. The body defines a hollow body 120 that encloses an internal compressible cavity 124. This cavity permits deformation and elastic recovery under gentle pressure, providing responsive tactile feedback. The body 102 incorporates a wall-thickness mapping 134A-C in which localized thickened regions correspond to structurally firm areas, while adjacent portions remain thinner and more compliant. Through this mapping, the biomimetic sensory engagement system 100 exhibits multiple tactile-firmness zones that collectively enhance oral, manual, and sensory engagement.

Positioned centrally in the figure is the handle loop 102, an integrally molded, fully enclosed gripping structure. The handle loop 102 is configured to be easily grasped by small hands from any direction, promoting early-stage grip training and fine motor skill development. Unlike rigid handles in prior approaches, the loop 102 is formed with a compliant wall thickness, allowing it to deform comfortably under baby grasp without compromising structural durability.

Above the handle loop 102 is the pacifier-shaped element 110, which forms a rounded, protruding structure designed to encourage oral engagement. This element 110 is positioned to align naturally with the baby’s mouth when the toy is held by the handle loop 102, creating a familiar, self-directed oral soothing experience. In contrast to prior teething solutions that utilize detachable pacifier components or hard plastics, the pacifier-shaped element 110 is integrally formed from solidified silicone through localized wall thickening, ensuring safe, repeated use without risk of detachment or material degradation.

Extending around the facial region and upward surface is a gum massager 108, presented as a mane-like array of firm prongs. The gum massager 108 is formed by increasing the wall thickness of the silicone in these localized regions, creating a series of soft-yet-rigid protrusions that remain flexible enough for safe chewing while providing tactile stimulation and pressure-based relief. This configuration avoids the need for embedded hard inserts, a drawback of certain prior approaches, and preserves full material homogeneity for ease of manufacturing and safety compliance.

The pacifier-shaped element 110 includes an integrally molded gum massager 108 formed as a ring or array of pronged projections. Each prong flexes independently during chewing, distributing gum pressure evenly and soothing erupting teeth. The gum massager 108 is produced through localized thickening of the silicone wall surrounding the pacifier element 110, eliminating separate inserts or adhesive joints and maintaining full monolithic integrity.

The exterior surface of the body 102 may include a visual indicator 112 in the form of a soothing visual indicator, such as a sleeping-eye indicator design molded or recessed directly into the silicone wall. The soothing visual indicator 112, also referred to as the sleeping-eye indicator 112, conveys a calming, restful aesthetic and doubles as a tactile tracing feature for developing fine-motor control. The soothing visual indicator 112 can be integrally formed during molding to prevent separation or contamination.

In an exemplary embodiment, adjacent to the pacifier element and gum massager are soothing visual indicators 112, illustrated as a pair of closed eyes molded directly into the exterior surface. These features serve not only as decorative character elements, but also as emotionally calming signals, reinforcing the toy’s intended association with rest, soothing, and non-disruptive play. Prior teething devices have largely ignored the role of visual cues in baby comfort; the inclusion of the soothing visual indicators 112 enhances emotional bonding and contributes to the toy’s multi-sensory benefit.

Located on the upper body and rear torso of the illustrated figure are the localized chew zones 104, which are areas of increased firmness produced through selective thickening of the wall structure. These zones are engineered to deliver a firmer chewing surface while maintaining surrounding softness, thereby creating a sensory contrast that sustains engagement. Unlike conventional teething devices that rely on interchangeable or detachable chewing elements, the present invention maintains these contrasting zones within a seamless body, reducing complexity and increasing user safety.

Scattered across various regions of the body are tactile texture zones 114, molded as shallow surface recesses or patterns. These features provide subtle, yet deliberate, tactile feedback as the baby explores the surface of the toy. The tactile texture zones 114 support sensory development and help stimulate exploratory behavior, differentiating the present invention from smooth-surfaced teething products that offer limited haptic stimulation.

Extending from the posterior region of the body 102 is a prehensile extension 106, preferably formed as a spiral tail. The extension 106 possesses a solid cross-section rigid body 122 and exhibits elastic memory, enabling it to wrap securely around a support structure 302, such as a stroller bar, a crib rail, or other suitable support. Upon release, the tail 106 uncoils and returns to its relaxed configuration without permanent deformation, allowing repeated use without loss of function.

Emerging from the rear of the toy is the prehensile feature 106, also referred to as a tail 106, which may be shaped in a spiral or nautilus-like configuration. This prehensile feature 106 is selectively thickened to be substantially solid and, in preferred embodiments, solid, allowing it to retain a curled shape under tension. The prehensile feature 106 is configured to wrap around external structures such as stroller bars, crib rails, or diaper bag loops. Once engaged, it holds securely due to the elastic memory inherent in the food-grade silicone material, and upon release, the tail returns to its original coiled form without permanent deformation. This enables attachment without clips, straps, or additional hardware, simplifying both use and cleaning while enhancing portability.

While the illustrated embodiment shows a spiral-shaped tail 106 for wrapping attachment, other configurations, such as a hook, loop, or S-curve, may be used. These alternative geometries preserve the elastic memory function while offering different modes of attachment or manipulation. In all configurations, the prehensile feature is designed to retain a coiled or fixed position without external fasteners, maintaining safety and ease of use.

At the bottom of the figure is the stabilizing base 118, configured to allow the biomimetic sensory engagement system 100 to rest in an upright position when placed on a flat surface. The wide, flat profile of the base enhances usability and hygiene by reducing the likelihood that oral contact regions will come into contact with contaminated surfaces. The base also contributes to the toy’s visual accessibility and upright storage between uses, solving a common frustration encountered with rolling or unstable teething toys in prior approaches.

The exemplary arrangement of components illustrated in FIG. 1 demonstrates how multiple sensory and ergonomic features can be incorporated into a single, monolithic form without introducing multiple materials, detachable elements, or external mounting hardware. By leveraging controlled variations in wall thickness and elastic behavior, the invention achieves a high degree of functionality while maintaining manufacturing simplicity, safety compliance, and baby usability.

In an exemplary embodiment, the toy is configured with character-like aesthetic features, including molded facial contours, ear-like side protrusions, and visual symmetry. These elements are not merely decorative, but serve a dual function of attracting and emotionally anchoring the baby user. Caregivers may attribute personality to the toy, and babies may associate the resting facial indicators with quiet or sleep time, supporting use during pre-nap soothing routines. The integration of such features reinforces user bonding and increases toy utility beyond purely functional teething relief.

In an exemplary embodiment, because the biomimetic sensory engagement system 100 can be molded as a single continuous body without seams or secondary assemblies, depending on the suitability of the materials, the biomimetic sensory engagement system 100 can be configured for repeated sterilization using boiling, steam, or dishwashing cycles, as may be required and/or desired in a particular embodiment. The absence of internal voids or unattached seams prevents microbial buildup, and the food-grade silicone maintains its structural integrity through repeated thermal cycling. This compatibility enhances the practical value of the invention for caregivers managing daily hygiene routines.

In an exemplary embodiment, to ensure upright stability on flat surfaces, the base 118 may include an increased wall thickness or molded-in material density at its lowest point. This structural massing produces a low center of gravity, helping the biomimetic sensory engagement system 100 return to an upright position after tipping. The weighted base also anchors the toy during baby interaction, preventing excessive rolling or unintended motion when placed on a high chair or changing table.

In an exemplary embodiment, the biomimetic sensory engagement system 100 can include a pair of ear-like projections 124 formed symmetrically on either side of the upper body. These projections are integrally molded and may function as auxiliary grip points, visual character enhancements, or additional textured chew regions. The ear like projections 124 are soft, compressible, and shaped to be safely engaged by baby hands or mouths without creating hard pressure points. These features further encourage bilateral manipulation and promote symmetrical hand use, an important developmental milestone.

In further reference to FIG. 1, the internal geometry of the biomimetic sensory engagement system 100 may be characterized by a wall-thickness mapping 120 that defines regions of differing rigidity and compressibility throughout the body. Thicker wall portions are associated with the pacifier-shaped element 110, the gum massager 108, and the localized chew zones 104, whereas thinner portions underlie the handle loop 102, tactile-texture zones 114, and the hollow torso cavity. This configuration yields progressive resistance during grasping or chewing, enabling infants to perceive distinct tactile gradients that reinforce oral-motor learning and spatial awareness. The variable-thickness construction is achieved within a single molding cycle and does not require inserts or multi-material bonds, thereby maintaining monolithic integrity while delivering differentiated sensory feedback.

In some embodiments, a sleeping-eye indicator 112 is recessed or molded in relief on both sides of the facial region. These indicators act as visual landmarks that support early recognition and emotional association, allowing caregivers to introduce the biomimetic sensory engagement system 100 as a calming or bedtime companion. The ear-like projections 124 may be texturized or perforated to introduce micro-acoustic effects when squeezed, providing a subtle sound component that complements tactile engagement.

When the prehensile feature 106 is wrapped about a rail or caregiver’s finger, the stabilizing base 118 cooperates to orient the biomimetic sensory engagement system 100 in a balanced, forward-facing position, presenting the pacifier-shaped element 110 outward for immediate oral access. The geometry thus emulates the natural head-to-tail alignment of small animals, reinforcing the biomimetic theme while ensuring ergonomic presentation to the child. This arrangement also facilitates convenient hanging for drying or storage, as gravity assists drainage from the hollow cavity through small airflow channels formed at the junction between the base 118 and torso region.

In certain embodiments, the biomimetic sensory engagement system 100 includes an integrated handle loop 104 disposed centrally along the body 102. The handle loop 104 is dimensioned for grasping by infant hands and provides a compliant gripping structure that deforms slightly under pressure yet returns to shape. The continuous molding of the handle loop 104 eliminates seams or attachment points, improving durability and washability.

In operation, a caregiver provides the biomimetic sensory engagement system 100 to a child such that the animal topology 140 is visually apparent. The child grasps the handle loop 104 or surrounding body 102, positions the pacifier-shaped element 110 for chewing or gum massage, and compresses the hollow body 120 to produce tactile or auditory feedback from the squeaker 116 when present. After use, the caregiver or child may wrap the prehensile extension 106 around a support 302 for hygienic drying or storage.

During normal operation, the child chews or gums the pacifier-shaped element 110, compresses the hollow body 120 to elicit tactile or audible feedback via the squeaker 116, and wraps the prehensile tail 106 about the support 302 for temporary suspension or drying between uses.

Collectively, the features illustrated in FIG. 1 demonstrate how localized geometry, visual design elements, and mechanical elasticity are cooperatively integrated within a single body to provide oral, tactile, and visual stimulation in a safe, hygienic, and developmentally beneficial form.

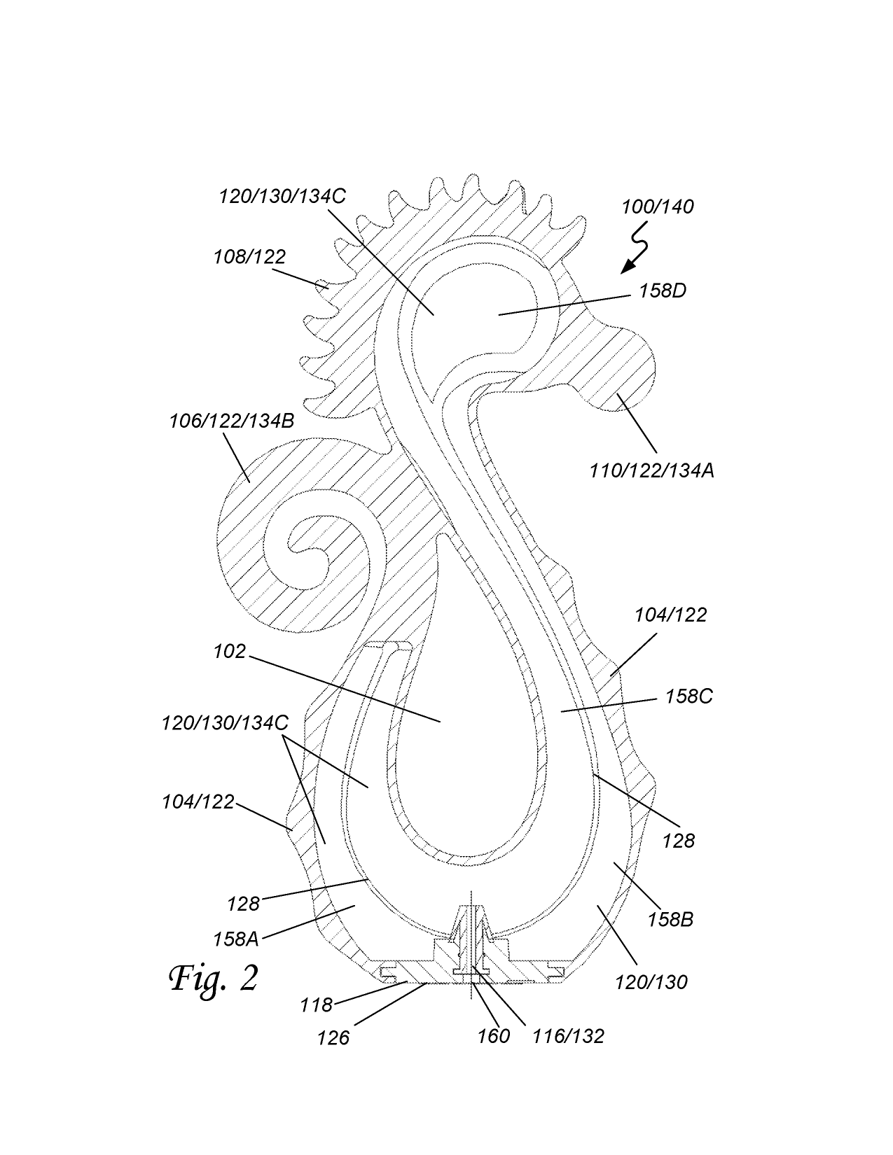

Referring to FIG. 2, there is illustrated one example of a cross-sectional side view of a biomimetic sensory engagement system 100. In an exemplary embodiment, the cross-section reveals the internal structure and material thickness distribution that defines the functional advantages of the present invention. The biomimetic sensory engagement system 100 is primarily composed of a hollow body 120 formed from a single molding of food-grade silicone, enabling compressibility, resilience, and acoustic functionality while maintaining structural safety and integrity.

The hollow body 120 surrounds an interior air volume that extends throughout the core of the system 100. This internal cavity allows the toy to deform under pressure—such as from baby gripping, chewing, or squeezing—while returning to its original shape due to the elastic memory of the silicone material. Unlike rigid or multi-piece teething devices of prior approaches, the hollow body construction contributes to both user comfort and dynamic responsiveness, supporting interactive engagement through sound, movement, and oral pressure feedback.

More specifically, in an exemplary embodiment, the internal structure of the hollow body 120 further defines a plurality of internal chambers 158A–158D that collectively manage airflow, pressure response, and acoustic behavior within the toy. These chambers are formed integrally during molding and are separated by continuous silicone partitions that maintain the monolithic integrity of the body. The chamber 158A comprises a tuned air channel 130 that fluidly interconnects with the orifice 126 located at the base of the toy. This chamber functions as a pressure-balancing conduit that equalizes internal air volume during compression and release, ensuring consistent tactile feedback and controlled air displacement. The chamber 158B also defines an air channel 130, but is sealed at both ends to form an enclosed, compressible volume that deforms resiliently under a child’s squeeze without collapsing. This configuration contributes to the toy’s soft, responsive feel and supports repetitive compression cycles without permanent deformation. The chamber 158C incorporates an air channel 130 acoustically coupled to an acoustic resonator 132, which amplifies and modulates the sound produced by the squeaker 116 when the toy is squeezed. This resonant chamber geometry enhances tonal clarity and volume while maintaining a gentle, non-startling sound profile appropriate for infant sensory development. The chamber 158D, positioned in the head region 136, defines another sealed air channel 130 that functions similarly to chamber 158B—remaining compressible under manual pressure but resisting full collapse. The distributed arrangement of these chambers 158A–158D provides localized zones of varying compliance and acoustic response throughout the hollow body 120, thereby enriching the multi-sensory experience by combining tactile, auditory, and kinesthetic feedback within a single, seamless structure.

The hollow body structure includes selectively thickened zones collectively referred to as the rigid body 122, which define the stiffer regions of the toy. These rigid sections are not separate parts but are formed integrally with the hollow body by locally increasing the wall thickness of the food-grade silicone. The rigid body 122 includes and supports structurally reinforced components such as the pacifier-shaped element 110, gum massager 108, localized chew zones 104, and prehensile feature 106. These zones retain their structural firmness even as the surrounding body remains compressible and pliable, enhancing both safety and function.

At the base of the hollow body is the squeaker element 116, which is embedded within the structure and acoustically coupled to the interior cavity. The squeaker element 116 is permanently enclosed within a molded housing and sealed against exposure, eliminating access to small parts or mechanical assemblies. When the baby compresses the body, air pressure within the hollow body 120 activates the squeaker element 116, producing an audible sound. This sound reinforces cause-and-effect learning and provides an additional sensory layer that sustains attention and promotes auditory development.

Encasing the squeaker is the stabilizing base 118, which is configured with a widened footprint and flat bottom surface to support upright positioning of the toy when at rest. The base both houses the acoustic feature and enables hygienic handling by minimizing contact between oral features and flat surfaces during storage or idle periods.

The cross-sectional view also illustrates the role of wall thickness variation in achieving regional functional zones. Specifically, multiple structures—including the pacifier-shaped element 110, gum massager 108, localized chew zones 104, and prehensile feature 106—are each shown to be supported by a corresponding section of the rigid body 122. These segments are formed by locally increasing the silicone wall thickness relative to surrounding areas. As a result, they exhibit enhanced stiffness and resistance to deformation, enabling those regions to function as firm chew targets or stable attachment structures.

The pacifier-shaped element 110, for instance, is positioned near the top of the toy and includes a solid cross-section, making it durable under repeated chewing and resistant to material fatigue. Its placement is ergonomically aligned with the grasping location of the handle loop 102, ensuring intuitive oral engagement by the baby. The underlying rigid body 122 in this region supports the pacifier structure and preserves its intended shape through cycles of compression.

The gum massager 108, illustrated here in cross-section, reveals the protruding, thickened prong-like structures that deliver massaging pressure during teething. Unlike conventional gum pads that rely on external inserts or textured overlays, this element is seamlessly formed with the rest of the toy. The rigidity of the rigid body 122 at the base of each prong ensures the massager maintains enough firmness to apply pressure while still being safe and comfortable.

The localized chew zones 104 located along the front and back torso regions also appear as reinforced segments within the hollow structure. These zones provide firmness and textural contrast during oral interaction, promoting continued exploration by the baby and satisfying the instinct to chew while teething. The rigid body 122 in these regions ensures that the chew zones do not collapse under moderate pressure, preserving the desired oral feedback.

The prehensile feature 106, depicted curling from the posterior of the toy, shows a solid internal section configured to wrap securely around crib rails, stroller handles, or other household structures. Unlike toys requiring separate attachment clips or fasteners, the prehensile tail is shaped and thickened to retain its curled position after wrapping. The corresponding segment of the rigid body 122 gives the tail sufficient stiffness to resist sagging and maintain functional repeatability after multiple uses.

Distributed along the outer shell are tactile texture zones 114, shown in the cross-sectional view as shallow depressions molded into the exterior wall. These texture features contribute to tactile diversity across the surface of the toy and encourage exploratory handling. The surface recesses are formed integrally during the molding process and do not compromise the structural or hygienic properties of the toy.

The cross-sectional view of FIG. 2 highlights one of the central innovations of the present invention: the ability to produce a complex, multi-functional toy from a single molding of uniform food-grade silicone, using localized thickening to control performance in specific regions. This approach avoids reliance on multiple materials or mechanical assemblies and instead achieves structural differentiation through precision in mold design. The resulting product is safe, durable, washable, and compliant with child safety standards—features that distinguish it from prior approaches, which often trade off functionality for manufacturability or require additional parts to deliver similar features.

In certain embodiments, the internal geometry of the hollow body 120 may include an acoustically tuned cavity surrounding the squeaker element 116. This design promotes resonance and consistent sound output when compressed, ensuring effective auditory feedback regardless of the toy’s orientation. The placement of the squeaker in the lower region of the toy also localizes the sound source away from the oral engagement areas, which helps preserve hygiene and reinforces audio-tactile associations during squeezing or chewing behavior.

In some configurations, the silicone wall thickness may be varied along a gradient to create a spectrum of firmness across the toy’s body. For example, the distal regions of the tail or limbs may remain more flexible, while central body regions offer medium resistance, and oral engagement areas such as the pacifier element and chew zones are formed as the most rigid. This progressive firmness gradient introduces stages of resistance that correspond to different developmental grasp strengths and oral pressures, thereby aligning with the evolving motor capabilities of babies across early growth stages.

In certain manufacturing methods, a temporary orifice 160 is left open at the base of the hollow body during compression molding to allow for placement of the acoustic resonator 132, squeaker element. Once the squeaker is securely inserted into the cavity, a silicone plug is positioned within the base opening and permanently sealed, either via heat bonding, chemical curing, or ultrasonic welding, creating a seamless, hygienic exterior. The sealing method is chosen to maintain the monolithic appearance and ensure structural continuity with the rest of the molded body.

In an exemplary embodiment, the geometry of the internal cavity, chamber 158C, and wall thickness 134A-C around the squeaker element are selectively tuned to modulate the pitch and volume of the emitted sound. These tuning variations enable the toy to produce consistent, non-startling auditory feedback suitable for baby auditory development. The acoustics may be optimized for activation under baby-level pressure ranges, providing reward-based learning through cause-and-effect engagement.

In an exemplary embodiment, the calming aesthetic of the visual indicator, particularly the sleeping-eye motif, positions the biomimetic sensory engagement system 100 as a transitional comfort object during nap or bedtime routines. The non-stimulating visual appearance encourages soothing rather than excitement, distinguishing it from visually overstimulating toys. Caregivers may use the toy as a ritualized calming item, fostering emotional regulation and association with restful periods.

In some embodiments, limb-like protrusions such as paws or foot structures are formed along the lower body to enhance the character's aesthetic and provide additional grasping zones or surface variation for oral or tactile engagement. These elements are soft and contoured to maintain safety while encouraging bilateral hand use.

In continued reference to FIG. 2, the sectional geometry further illustrates how localized variations in wall-thickness mapping 134A-C cooperate with the internal cavity of the hollow body 120 and air chambers 158A-D, which can be configured as tuned air channels 130, to create distinct mechanical responses under compression. With regards to the wall-thickness mapping 134A-C, the region 134A immediately adjacent to the pacifier-shaped element 110 exhibits the highest stiffness values, producing firm oral resistance suitable for gum-soothing engagement. Transitional regions 134B and 134C between the rigid body 122 and the thinner peripheral walls act as compliant diaphragms that distribute pressure evenly throughout the cavity, enhancing durability and preventing material fatigue after repeated chewing cycles.

The prehensile feature 106, head region 136, and torso region 138 can include an optional longitudinal reinforcement rib 128 embedded within the silicone matrix during molding. This reinforcement rib 128 segments tuned air channels 130, and provides directional stiffness along the curl axis, enabling the tail to retain its wrapped configuration for longer durations without sagging. When the feature 106 is extended and released, the stored elastic energy produces a gentle return motion that contributes to the tactile and visual engagement of the child, mimicking organic tail movement found in small animals and reinforcing the biomimetic character of the system 100.

The acoustic chamber surrounding the squeaker element 116 may include chamber 158C configured as a tuned air channel 130 formed along the inner wall of the hollow body 120. These channels modulate air flow to generate a controlled amplitude range, ensuring that the emitted sound remains within safe auditory levels for infants while still perceptible enough to sustain attention. The air channels also facilitate drying of the interior cavity after washing, promoting hygiene and preventing moisture entrapment around the squeaker housing.

Proximate the upper torso region 138, the soothing visual indicator 112 is visible in the sectional profile as a recessed contour whose depth corresponds to the surrounding wall-thickness mapping 134C. The integration of the indicator within a thickened region enhances mold fidelity and prevents thin-wall distortion during manufacturing. In use, the indicator functions as both a calming visual symbol and a tactile depression that infants may trace with their fingers, linking visual recognition with fine-motor feedback.

In an exemplary embodiment, the biomimetic sensory engagement system 100 may implement a three-region wall-thickness profile 134A–134C defining a first region 134A of greatest wall thickness forming the oral-engagement zone 110, a second region 134B of intermediate thickness forming the prehensile tail 106, and a third region 134C of reduced thickness forming the compressible torso 120. These graduated zones create a localized firmness gradient that aligns with the functional load each region experiences during use.

In some configurations, an alternate handle loop 104A may be positioned laterally or seamlessly blended within the contour of the body 102. The loop 104A maintains ergonomic symmetry with the pacifier-shaped element 110 and the prehensile tail 106 to preserve the overall biomimetic appearance 140.

Together, these cross-sectional relationships reveal how the biomimetic sensory engagement system 100 achieves multidimensional performance through monolithic molding alone. The deliberate coupling of mechanical elasticity, acoustic resonance, and visual-tactile signaling within a single silicone body provides a unified developmental experience for the infant user, while preserving manufacturability, safety, and aesthetic integrity.

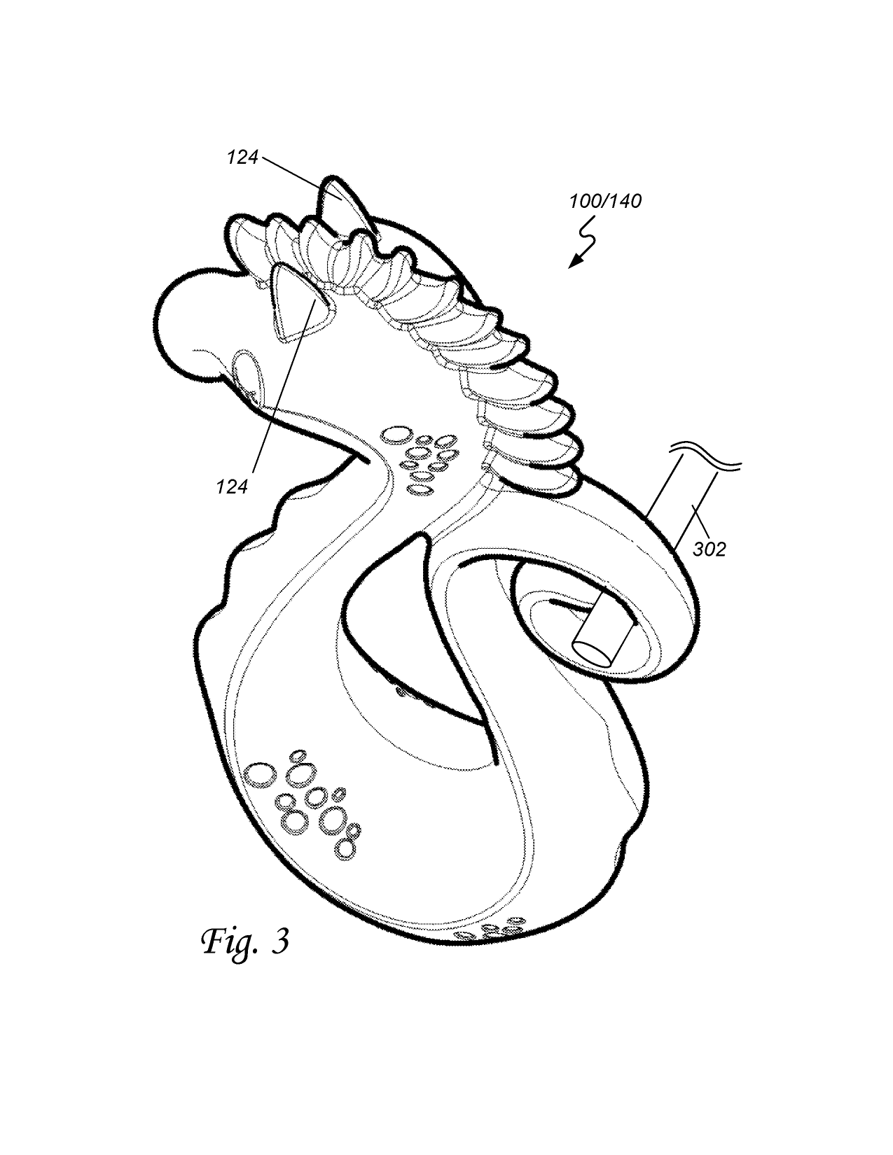

Referring to FIGS. 3-6, there are illustrated various perspective views of the biomimetic sensory engagement system 100, a toy for use by a child. In an exemplary embodiment, these views demonstrate the unified integration of the toy’s ergonomic shape, surface features, and functional contours. From different angles, the structural relationship among the handle loop, pacifier-shaped element, gum massager, prehensile tail, chew zones, and stabilizing base can be appreciated. These perspective illustrations also highlight the smooth transitions between functional zones, reinforcing the toy’s single-material, monolithic construction. The views emphasize the compact footprint and overall portability of the device, which are key benefits over bulkier or multi-piece teething toys of prior approaches.

In further reference to FIGS. 3-6, the multiple perspective views collectively illustrate the three-dimensional continuity of the biomimetic sensory engagement system 100. From these orientations, the seamless flow between functional zones becomes apparent: the pacifier-shaped element 110 projects forward along the longitudinal axis, merging smoothly into the surrounding gum massager 108 and the adjacent localized chew zones 104, each supported by a corresponding region of the rigid body 122. These thicker regions transition gradually into the thinner, more compliant sections defining the handle loop 102 and central torso, evidencing the system’s integrated wall-thickness mapping 124. This continuity of geometry enables the body to deform and recover uniformly under load, eliminating stress points that could otherwise occur at joints or seams.

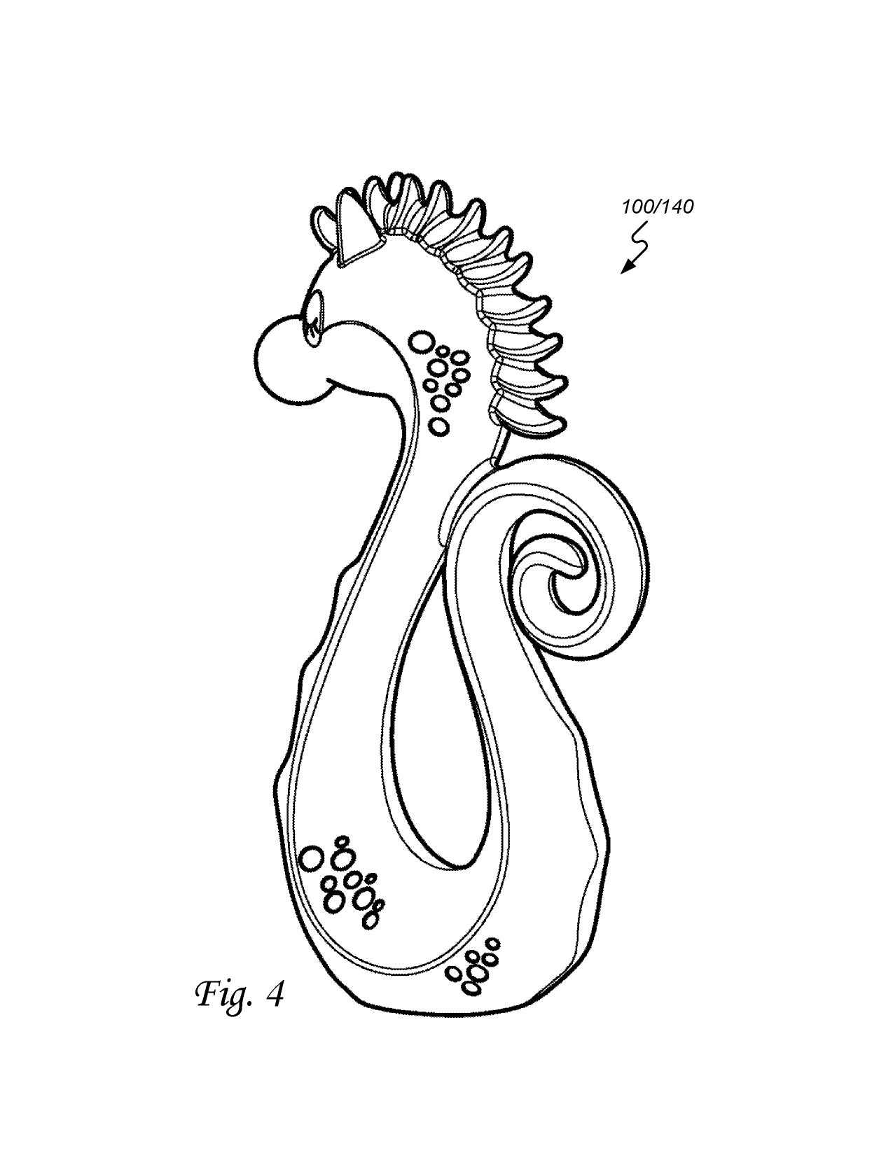

The spatial arrangement shown in FIGS. 3-4 demonstrates how the handle loop 102 and prehensile feature 106 cooperate to provide dual-mode handling. When grasped by the handle loop 102, the pacifier-shaped element 110 aligns naturally with the child’s oral axis for intuitive use. When suspended by the prehensile feature 106, the loop 102 serves as a stabilizing counterbalance, maintaining a forward-facing orientation that keeps oral regions accessible and elevated from contact surfaces. This dual functionality allows the biomimetic sensory engagement system 100 to act both as a hand-held device and as a hanging developmental accessory.

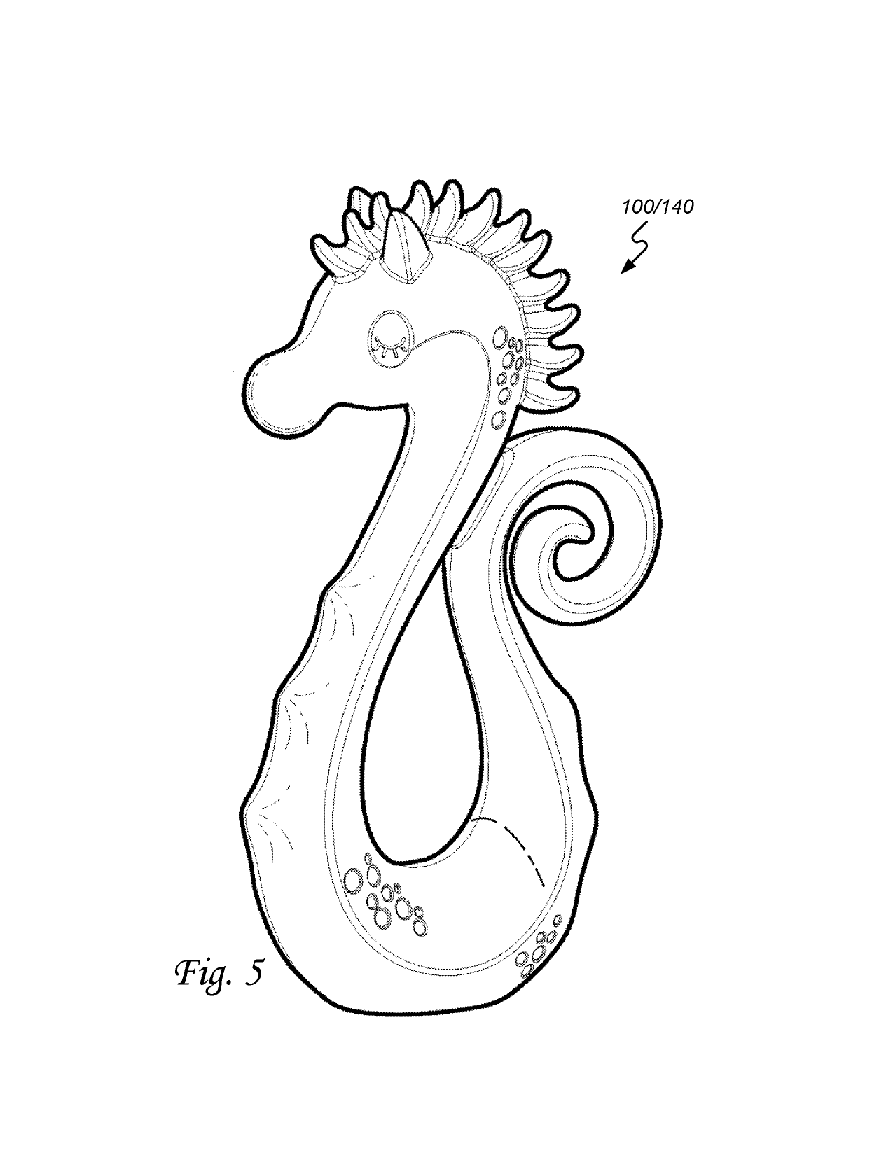

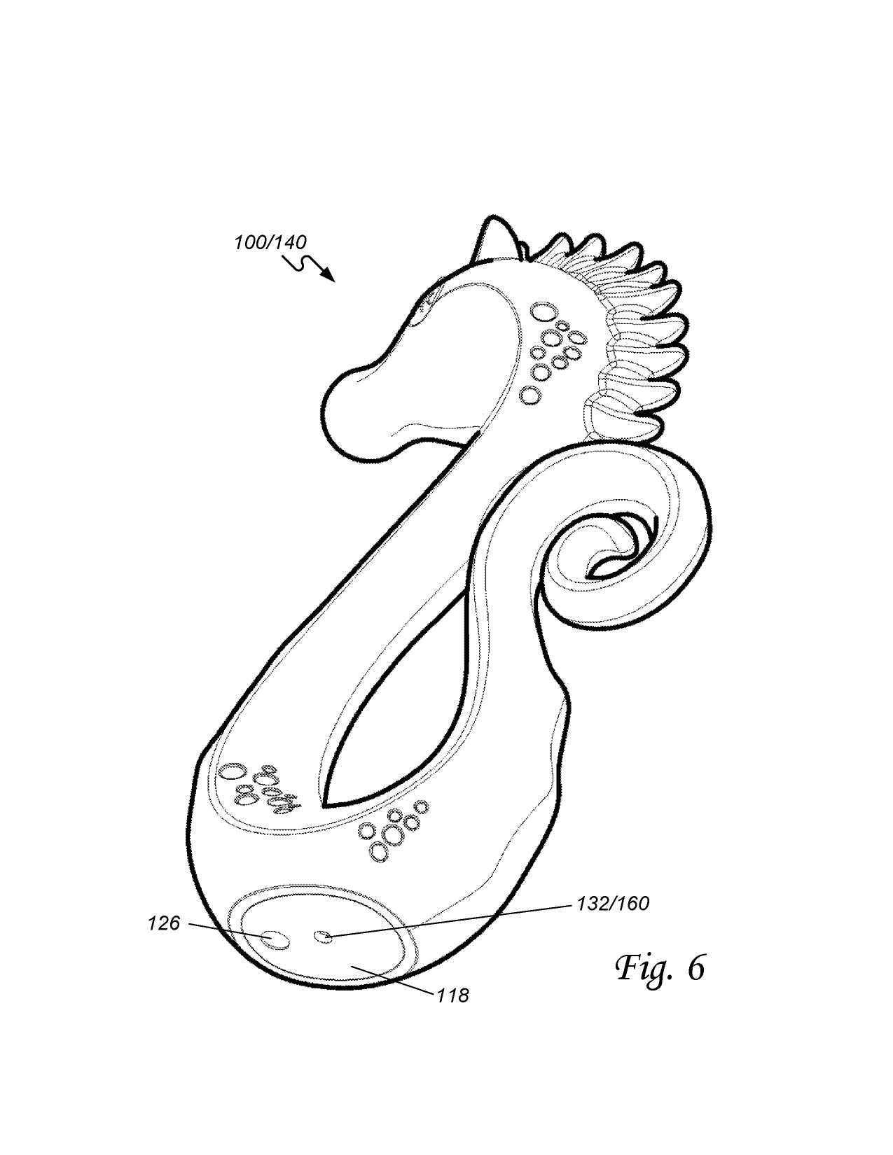

As seen in FIGS. 5-6, the outer contours form a recognizable biomimetic topology reminiscent of small animals such as a seahorse. Other recognizable biomimetic topologies reminiscent of small animals can include monkey, turtle, dolphin, or other animals, some of which are better illustrated in at least FIG. 11. The curvature of the prehensile feature 106 represents the tail region, while the pacifier-shaped element 110 defines the head or snout portion. The proportional distribution of these regions provides visual familiarity that naturally attracts infant attention and encourages voluntary engagement. The soothing visual indicator 112, visible on each lateral surface, reinforces the perception of a peaceful, resting creature, promoting emotional comfort during teething or quiet play periods.

The perspective views also reveal the distributed pattern of tactile-texture zones 114, which vary in density and geometry across different surfaces to create graduated haptic responses. The front torso region may feature broader ridges for palm contact, while the dorsal and limb-like areas incorporate finer textures for fingertip exploration. This heterogeneity of texture strengthens sensory differentiation and supports progressive fine-motor development as the child learns to manipulate and rotate the biomimetic sensory engagement system 100.

From a manufacturing standpoint, FIGS. 3-6 emphasize how ergonomic contours, textural detailing, and internal wall-thickness variations are achieved within a single-mold process without secondary assembly. The resulting structure maintains consistent durometer and tensile performance throughout, ensuring compliance with infant safety standards while reducing material waste and production complexity. Collectively, these perspective views confirm that the biomimetic sensory engagement system 100 integrates aesthetic harmony, developmental functionality, and structural efficiency within one unified form, distinguishing it from prior teething devices.

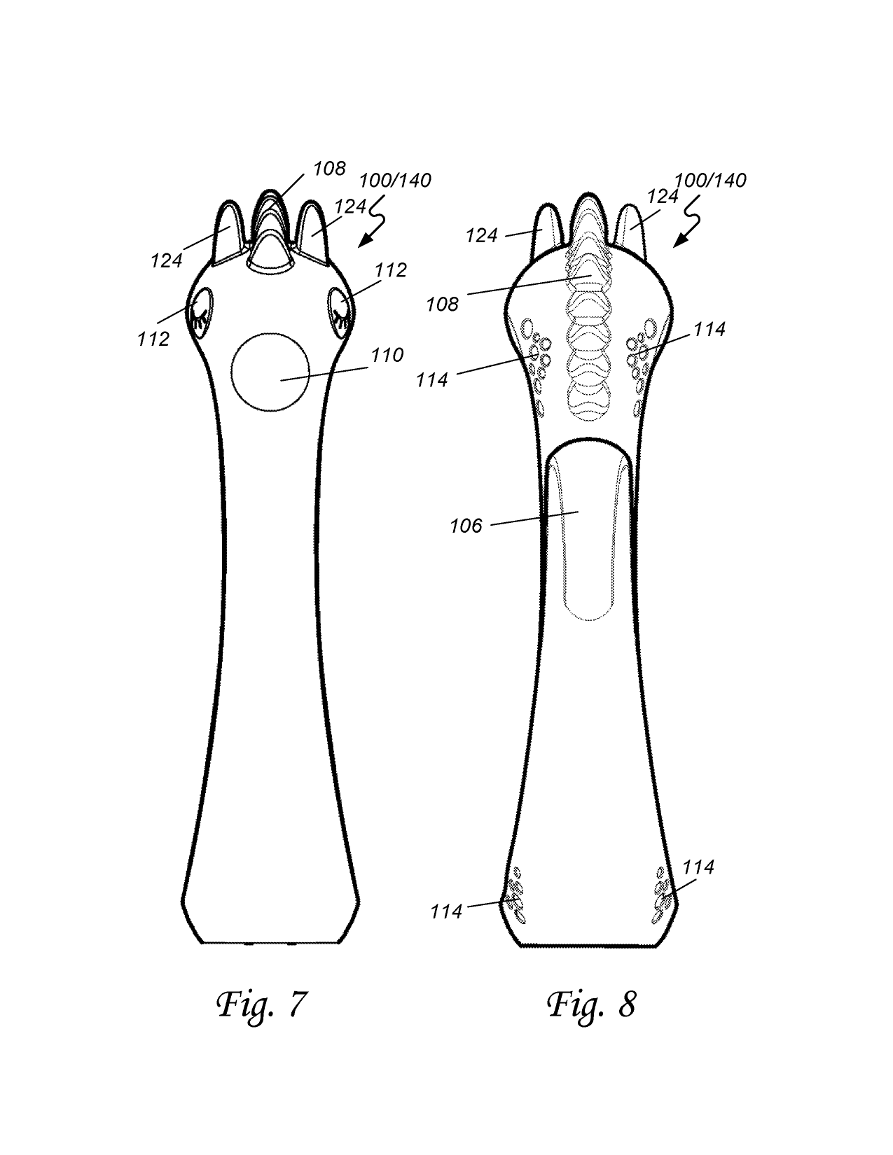

Referring to FIG. 7, there is illustrated a front view of the biomimetic sensory engagement system 100, a toy for use by a child, showing the arrangement and visual styling of the gum massager and pacifier-shaped element. In this orientation, the sleeping-eye soothing indicators 112 are prominently visible, further supporting the toy’s emotional calming functionality. The view underscores the symmetrical layout and intuitive orientation for baby engagement.

In continued reference to FIG. 7, the frontal orientation illustrates the deliberate bilateral symmetry of the biomimetic sensory engagement system 100, which facilitates intuitive alignment and predictable handling by infants who are still developing left–right coordination. The centrally positioned pacifier-shaped element 110 defines the system’s visual focal point, projecting forward along the longitudinal axis and framed by the surrounding gum massager 108 formed as a concentric ring of soft prongs. This configuration establishes a balanced distribution of oral-engagement surfaces that can be approached from either side, enabling self-initiated mouthing without the need for rotational orientation.

Flanking the pacifier element are the sleeping-eye soothing indicators 112, recessed slightly into the outer wall to provide both visual and tactile definition. The molded depth of each indicator is selected to create gentle shadow contrast under ambient light, enhancing visual recognition even for early-stage visual acuity in infants. The indicators also serve as tactile depressions that the child may trace with fingertips, linking visual and proprioceptive feedback in a calming, repetitive motion that reinforces emotional regulation.

The front view further reveals the distribution of tactile-texture zones 114 across the cheeks and upper torso portions of the biomimetic sensory engagement system 100. These zones form micro-patterns that transition outward from the central gum-massager region 108 to peripheral body surfaces, establishing a gradient of surface roughness that supports exploratory touch. The progression from smooth to textured surfaces provides a sensory “map” that encourages longer engagement periods and fine-motor exploration.

From a manufacturing standpoint, the front-facing symmetry ensures uniform silicone flow within the mold cavity during injection or compression molding, reducing air entrapment and promoting consistent wall-thickness mapping 134A-C across both sides of the device. This uniformity contributes to mechanical balance during use, ensuring that compressive forces applied during chewing or squeezing are evenly distributed through the hollow body 120.

The visual composition of FIG. 7 also conveys the biomimetic character of the biomimetic sensory engagement system 100, resembling the calm, resting expression of a small animal creature. This recognizable topology naturally invites interaction while communicating safety and comfort through visual familiarity. The integration of these emotional design cues with functional oral and tactile zones exemplifies the holistic approach of the present invention—uniting structural engineering, developmental psychology, and sensory science within a single monolithic form.

Referring to FIG. 8, there is illustrated a back view of the biomimetic sensory engagement system 100, where the full form of the prehensile tail 106 is visible. This view highlights the curvature and alignment of the tail with the body, showing how the design supports wrapping around external fixtures for hanging or storage. It also reveals how the back chew zones and tactile features are integrated without seams or surface interruptions.

In continued reference to FIG. 8, the rear perspective of the biomimetic sensory engagement system 100 illustrates how the prehensile feature 106 integrates with the surrounding body to form a continuous curvature that originates at the posterior torso and terminates in a tapered end. The cross-sectional contour of the prehensile feature 106 is thickened along its central axis and gradually transitions to thinner lateral walls, a configuration derived from the system’s wall-thickness mapping 124. This gradient in stiffness allows the tail to bend and coil under moderate force yet retain sufficient rigidity to sustain its wrapped configuration once engaged around a support structure such as a crib rail, stroller handle, or caregiver finger. When released, the inherent elastic memory of the silicone material restores the original curvature, enabling repeated attachment without material fatigue or deformation.

The back view also reveals the placement of the localized chew zones 104 positioned along the dorsal torso and extending toward the tail base. These regions exhibit increased firmness relative to surrounding areas and are contoured to mirror the natural arch of the body, creating a stable biting surface that complements the front-side chew regions. The symmetrical distribution of these chew zones ensures that the child can engage with the biomimetic sensory engagement system 100 from any direction, enhancing usability and prolonging interaction time.

Distributed across the rear surface are tactile-texture zones 114, molded as a series of shallow ridges and dimples that introduce variable haptic feedback during grasping or rubbing. These rear textures differ slightly in pattern from those on the anterior side, providing a contrasting tactile experience that reinforces sensory differentiation. The pattern orientation follows the curvature of the prehensile feature 106, visually guiding the child’s exploration along the tail and helping to develop fine-motor tracking and bilateral coordination.

The curvature alignment shown in FIG. 8 also demonstrates the ergonomic and hygienic advantages of the biomimetic configuration. When the prehensile feature 106 is wrapped around a support, the body of the biomimetic sensory engagement system 100 naturally hangs in a downward orientation that prevents contact between the pacifier-shaped element 110 and potentially contaminated surfaces. This suspended posture allows for air circulation through the hollow body 120, expediting drying after hand-washing and maintaining a sanitary condition between uses.

From an aesthetic perspective, the rear silhouette evokes the anatomy of a small creature—such as a seahorse, monkey, dolphin, turtle, or other animal—reinforcing the invention’s biomimetic identity and strengthening the emotional connection perceived by both child and caregiver. The combination of sculpted curvature, continuous wall-thickness transitions, and integrated texture patterning underscores the unified, monolithic construction of the biomimetic sensory engagement system 100. Together, these features demonstrate how functional engineering and developmental psychology converge within a single molded form to create a safe, versatile, and emotionally resonant sensory engagement device.

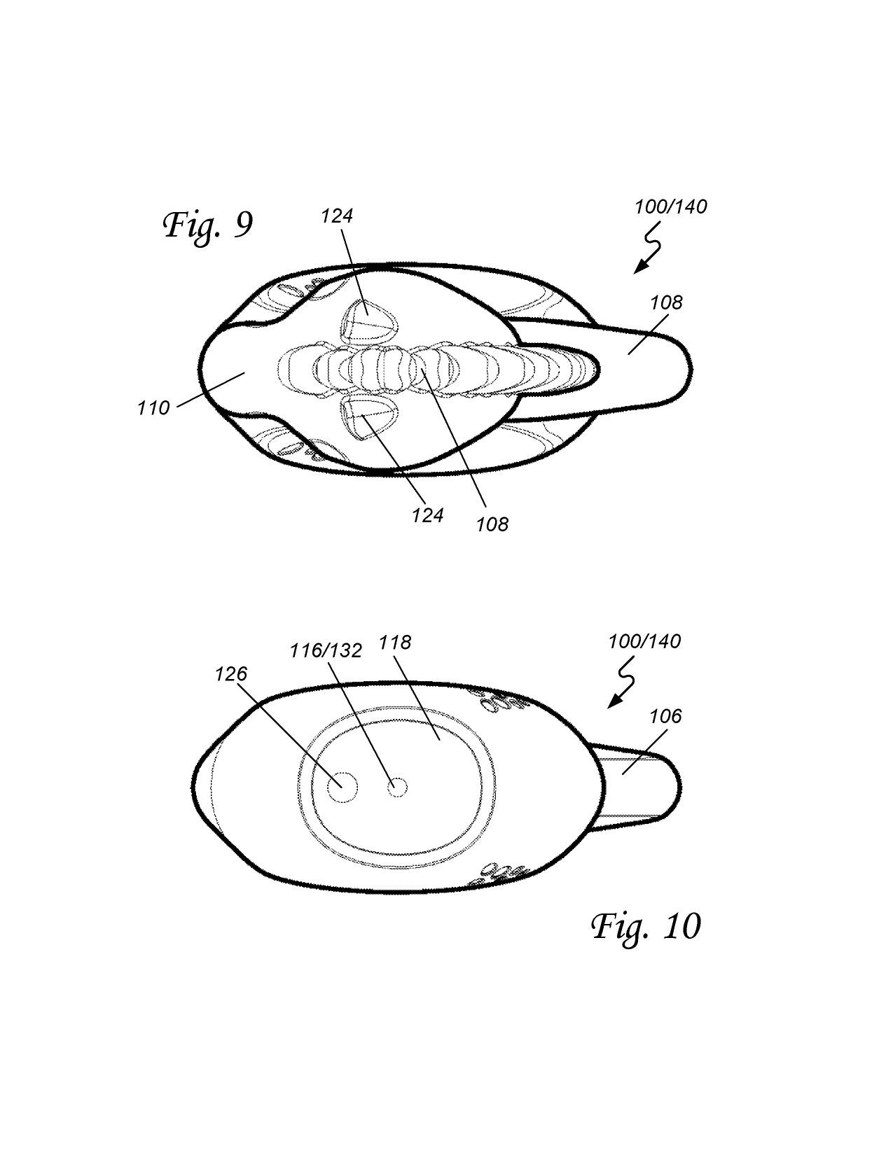

Referring to FIG. 9, there is illustrated a top view of the biomimetic sensory engagement system 100, emphasizing the spatial relationship between the gum massager prongs, pacifier-shaped element, and upper tactile regions. This orientation also reflects the visual appeal of the toy’s character-like features, including the rounded facial structures that invite oral engagement.

In continued reference to FIG. 9, the top view of the biomimetic sensory engagement system 100 provides a clear visualization of the radial symmetry and surface continuity between the gum massager 108, the pacifier-shaped element 110, and the adjacent tactile-texture zones 114. The gum massager 108 is formed as a circumferential array of prong-like structures radiating outward from the pacifier-shaped element 110, each prong being integrally molded with a localized increase in wall thickness to provide firm yet elastic resilience during chewing. The arrangement produces an alternating pattern of raised and recessed contours that generate multi-directional pressure variations against the gums, offering both soothing and stimulating tactile effects that promote oral–motor development.

From this overhead perspective, the sleeping-eye indicators 112 are shown in bilateral alignment across the upper portion of the structure, reinforcing the toy’s character-like facial impression. Their recessed geometry creates subtle shadows that enhance contrast under natural lighting, aiding early-stage visual detection and reinforcing emotional association. The spacing and curvature of these indicators are engineered to maintain material flow balance during molding, ensuring even wall thickness beneath the facial surface and preventing distortion during demolding or cooling.

The tactile-texture zones 114 visible around the crown and upper torso areas exhibit fine micro-patterning that transitions toward smoother surfaces near the pacifier-shaped element 110. This gradient texture provides a natural cue for the infant to transition between oral and manual exploration, thereby integrating multiple sensory modalities within a single interaction. When viewed from above, these textures form a cohesive aesthetic motif that enhances the visual biomimicry of a calm, resting creature.

The top view also reveals how the wall-thickness mapping 134A-C supports mechanical uniformity across the entire head region of the biomimetic sensory engagement system 100. The silicone material thickens beneath the gum massager 108 and pacifier-shaped element 110 to resist collapse under compression, while the peripheral crown area remains thinner to promote responsive deformation during gripping or squeezing. This intentional modulation of thickness provides functional hierarchy within the structure—firmness where support is required and softness where flexibility benefits the user experience.

From a usability standpoint, the broad, rounded contours observed in FIG. 9 allow the biomimetic sensory engagement system 100 to comfortably interface with the infant’s face and hands without sharp transitions or abrupt edges. The smooth curvature, combined with the integrated handle loop 102 positioned below, enables natural self-orientation and minimizes accidental poking or discomfort. The top view thus captures not only the structural elegance of the system’s design but also the thoughtful interplay between safety, sensory stimulation, and visual familiarity—attributes that collectively distinguish the present invention from conventional teething aids.

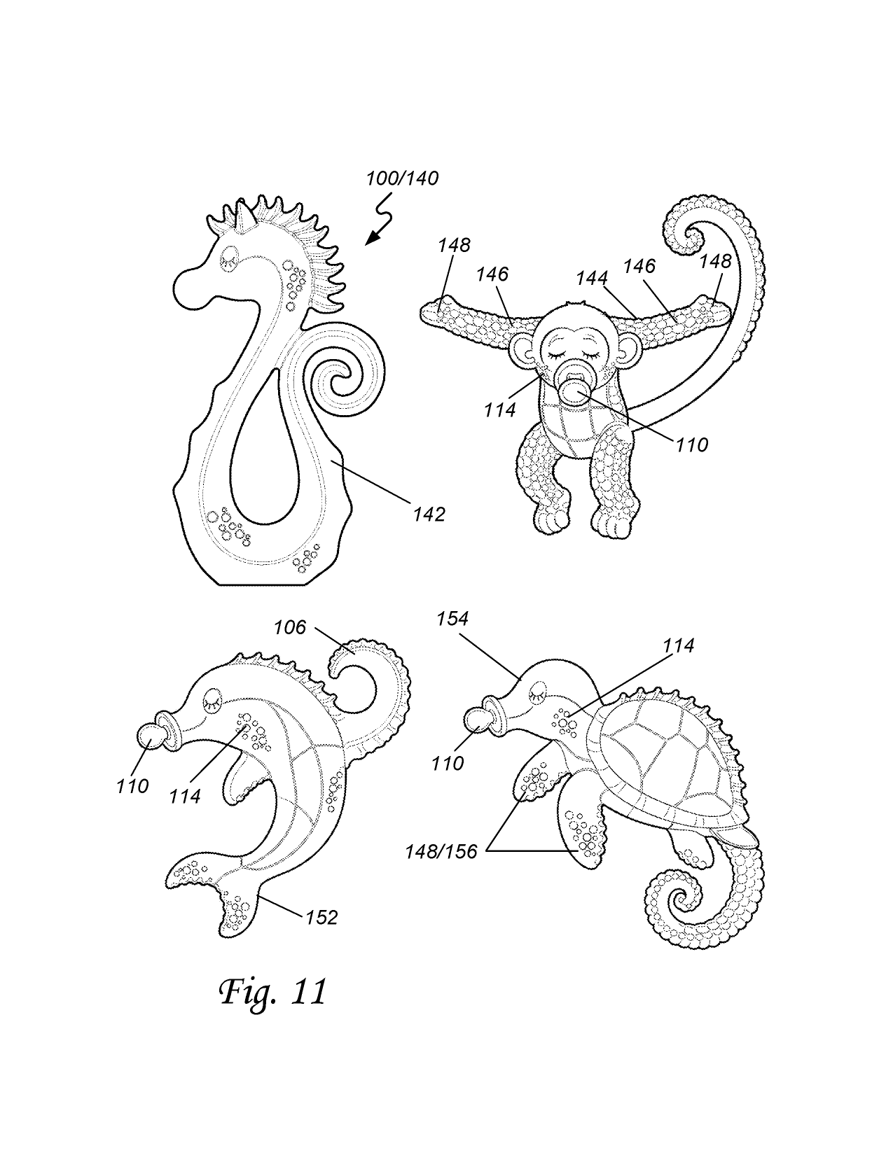

Referring to FIG. 10, there is illustrated a bottom view of the biomimetic sensory engagement system 100, clearly showing the wide, flat stabilizing base. In this view, the location of the embedded squeaker and its integration within the sealed structure can be inferred. The base design is shown to promote upright placement while maintaining the hygiene of elevated oral-contact features, further differentiating this design from prior approaches that tend to rest in unstable or contamination-prone orientations.

In continued reference to FIG. 10, the bottom view of the biomimetic sensory engagement system 100 illustrates the geometry and internal arrangement of the stabilizing base 118 in relation to the hollow body 120 and squeaker element 116 housed therein. The stabilizing base 118 is characterized by a widened footprint and low-profile curvature that collectively lower the center of gravity of the device, allowing it to remain upright during placement or minor disturbances. This base configuration enhances hygiene by ensuring that the pacifier-shaped element 110, gum massager 108, and other oral-contact surfaces remain elevated from the resting surface. Such elevation minimizes microbial transfer and prevents accidental contact with contaminated areas, an important improvement over conventional teething devices that roll or rest on their oral regions.

In the illustrated embodiment, the base 118 encloses a hermetically sealed compartment in which the squeaker element 116 is permanently embedded. The interior cavity of the hollow body 120 is acoustically coupled to the squeaker 116 through a set of chambers 158A-D configured as molded airflow channels 130, visible as narrow pathways radiating from the central cavity toward the base region. These channels allow air compression during squeezing or chewing to activate the squeaker without creating fluid ingress routes, thereby preserving both functionality and hygiene. The enclosure is fully sealed by a molded silicone membrane that is integrally bonded to the surrounding base walls during manufacturing, eliminating detachable plugs or mechanical joints that could trap moisture or debris.

The bottom view also reveals the concentric wall-thickness mapping 134A-C used to reinforce the base region. The peripheral rim of the base 118 exhibits an increased thickness relative to the central diaphragm area, producing a subtle flex zone that absorbs impact if the toy is dropped while maintaining stable footing during play. This graded wall structure contributes to both mechanical durability and acoustic performance by damping unwanted vibrations that could distort sound emission from the squeaker 116.

In some embodiments, small vent apertures 126 or acoustic resonators 116/132 may be integrally molded along the underside of the base 118 to fine-tune sound amplitude and tone. These apertures are dimensioned to remain imperceptible to the child’s touch yet serve to equalize air pressure during compression and release cycles. The resulting sound is soft, consistent, and calibrated to infant-safe decibel levels, reinforcing cause-and-effect learning without startling the child.

From a functional perspective, the base 118 also facilitates post-use drying and storage. When the biomimetic sensory engagement system 100 is placed upright on a countertop or tray, any residual moisture drains downward toward the base area, where airflow around the outer rim promotes evaporation. The upright orientation also preserves visual appeal by maintaining the recognizable biomimetic posture of the device, which may resemble a small resting animal, such as a seahorse, dolphin, or other animal.

Manufacturing efficiency is further supported by the bottom geometry illustrated in FIG. 10. The continuous curvature of the base allows for single-direction mold release, reducing tooling complexity and ensuring consistent silicone flow during injection or compression molding. The embedded squeaker 116 and tuned air channels 130 can be installed and sealed in-line without secondary assembly steps, resulting in a fully integrated, monolithic product that complies with child safety standards while delivering multi-sensory performance.

In use, the caregiver provides the biomimetic sensory engagement system 100 to the child so that the oral-engagement portion 110 corresponding to the head region 136 is accessible. The child chews or gums this region, compresses the hollow torso 120 to activate the squeaker element 116, and receives simultaneous oral, tactile, and auditory stimuli. These interactions reinforce recognition of the biomimetic form 140 and support cognitive and motor development while maintaining hygienic safety.

In combination, the features shown in FIG. 10 underscore the thoughtful integration of acoustic, structural, and hygienic engineering within the biomimetic sensory engagement system 100. The stabilizing base 118, reinforced by wall-thickness mapping 134A-C and enclosing the squeaker element 116, provides the foundation for a safe, self-supporting, and engaging developmental device that unites tactile, auditory, and visual stimuli within one continuous, child-friendly form.

Referring to FIG. 11, there are illustrated recognizable biomimetic topologies 140. In a plurality of exemplary embodiments, the spatial arrangement of the pacifier-shaped element 110, body 102, and prehensile tail 106 defines a recognizable biomimetic topology 140 corresponding to an animal figure. In a seahorse configuration 142, the curled tail forms the prehensile extension 106, while the pacifier-shaped element 110 represents the snout. In a monkey configuration 144, limb-like protrusions 146 extend from the sides of the body 102 to form auxiliary chew zones 148, enhancing tactile diversity while reinforcing the animal-like silhouette.

In an alternate embodiment, the biomimetic sensory engagement system 100 comprises a monolithic body 102 integrally defining multiple functional regions that cooperate to create a unified developmental device. The structure includes a pacifier-shaped oral-engagement portion 110 having a localized chew zone 108, a hollow torso cavity 120 that forms a deformable internal volume, and a prehensile tail 106 configured for self-wrapping attachment around external supports. Each of these regions is molded as part of a single, continuous body of food-grade silicone and contributes distinct sensory and ergonomic functions while maintaining overall monolithic integrity.

The relative positioning of the oral-engagement portion 110, torso cavity 120, and prehensile tail 106 emulates the head region 136, torso region 138, and tail anatomy 150 of an animal, producing a recognizable biomimetic topology 140 that naturally invites visual and tactile interaction by a child. This spatial organization promotes instinctive engagement, as the recognizable head-to-tail alignment provides a familiar and emotionally comforting form.

Representative examples of this embodiment include a dolphin configuration 152, wherein the prehensile tail 106 is shaped as a dorsal hook for hanging or storage, a turtle configuration 154, in which leg-like protrusions 156 extend laterally to create auxiliary chew zones 148, and variations in tactile-texture zones 114 that mimic anatomical surface features such as scales, ridges, or skin folds. These sculpted surface characteristics enhance realism and provide diverse haptic stimulation, reinforcing the child’s sensory exploration and developmental benefit while preserving the seamless, hygienic construction of the monolithic body 102. In further embodiments, other animal-inspired configurations incorporating the described biomimetic features—such as varying prehensile appendages, chew zone placements, and tactile-texture distributions—may likewise be realized within the scope of the present invention.

While the preferred embodiment of the invention has been described, it will be understood that those skilled in the art, both now and in the future, may make various improvements and enhancements which fall within the scope of the claims which follow.

These claims should be construed to maintain the proper protection for the invention first described.

What is claimed is:

1. A biomimetic sensory engagement system configured to promote oral, tactile, and visual interaction by a child, the system comprising:
a unitary body formed of food-grade silicone having a hollow interior defining a compressible volume;
a pacifier-shaped element protruding from a first region of the body and defining an oral-engagement region;
a prehensile extension configured to wrap around a support structure and to retain its wrapped configuration through elastic memory of the silicone material; and
at least one tactile-texture zone disposed on an exterior surface of the body;
wherein the oral-engagement region and the prehensile extension are spatially arranged along a longitudinal axis of the body to define a recognizable biomimetic form corresponding to an animal, such that the recognizable form encourages voluntary engagement by a child through recognition of the animal-like topology.

2. The biomimetic sensory engagement system of claim 1,
wherein the body comprises a wall-thickness mapping in which regions associated with oral engagement are thicker and more rigid than adjacent hollow regions, thereby producing multiple zones of tactile firmness that collectively enhance sensory engagement.

3. The biomimetic sensory engagement system of claim 1, wherein the oral-engagement region includes a gum-massager comprising pronged elements integrally molded with the pacifier-shaped element.

4. The biomimetic sensory engagement system of claim 1, wherein the prehensile extension comprises a spiral tail having a solid cross-section and elastic memory configured for self-wrapping attachment.

5. The biomimetic sensory engagement system of claim 1, further comprising a handle loop integrally formed with the body and dimensioned for baby grasping.

6. The biomimetic sensory engagement system of claim 1, wherein the recognizable biomimetic form corresponds to a seahorse configuration having a spiral tail forming the prehensile extension.

7. The biomimetic sensory engagement system of claim 1, wherein the recognizable biomimetic form corresponds to a monkey configuration having limb-like protrusions defining auxiliary chew zones.

8. A method of using the biomimetic sensory engagement system of claim 1, the method comprising the steps of:
providing the system to a child such that the recognizable animal topology is visually apparent;
allowing the child to grasp the body or handle loop (when present) and position the pacifier-shaped element for chewing or gum massage;
permitting the child to compress the hollow body to generate tactile or auditory feedback; and
attaching the prehensile extension around a support structure to store or suspend the system when not in use;
whereby the recognizable biomimetic form encourages voluntary engagement by the child through recognition of the animal-like topology.

9. A biomimetic sensory engagement system configured to promote oral, tactile, and visual interaction by a child, the system comprising:
a unitary body formed of food-grade silicone having a hollow interior defining a compressible volume;
a pacifier-shaped element protruding from a first region of the body and defining an oral-engagement region formed by a region of increased wall thickness relative to adjacent portions of the body;
a prehensile extension configured to wrap around a support structure and to retain its wrapped configuration through elastic memory of the silicone material; and
at least one tactile-texture zone disposed on an exterior surface of the body;
wherein the oral-engagement region and the prehensile extension are spatially arranged along a longitudinal axis of the body to define a recognizable biomimetic form corresponding to an animal, and wherein the body comprises a wall-thickness mapping in which regions associated with oral engagement are thicker and more rigid than adjacent hollow regions, thereby producing multiple zones of tactile firmness that collectively enhance sensory engagement.

10. The biomimetic sensory engagement system of claim 9, wherein the wall-thickness mapping defines three regions of differing rigidity, including:
a first region of greatest wall thickness forming the oral-engagement zone,
a second region of intermediate wall thickness forming the prehensile extension, and
a third region of reduced wall thickness forming a compressible torso portion.

11. The biomimetic sensory engagement system of claim 9, further comprising a handle loop integrally formed with the body and dimensioned for baby grasping.

12. The biomimetic sensory engagement system of claim 9, wherein the unitary body is monolithic and free of seams, joints, or detachable parts and is washable by hand or dishwasher without degradation.

13. The biomimetic sensory engagement system of claim 9, wherein the unitary body comprises a visual indicator formed as a recessed or molded sleeping-eye design configured to promote calming visual association during infant use.

14. A method of using the biomimetic sensory engagement system of claim 9, the method comprising the steps of:
allowing a child to chew or gum the pacifier-shaped element of increased wall thickness;
compressing the body to generate tactile or acoustic response; and
wrapping the prehensile extension about a support to store or suspend the system between uses.

15. A biomimetic sensory engagement system configured to promote oral, tactile, and visual interaction by a child, the system comprising:
a monolithic body formed of food-grade silicone and integrally defining:
a pacifier-shaped oral-engagement portion having a localized region of increased wall thickness that defines a chew zone;
a hollow torso portion providing an internal compressible cavity; and
a prehensile tail configured to elastically wrap around a support;
wherein the oral-engagement portion and the prehensile tail are positioned to emulate anatomical head and tail regions of an animal, thereby defining a recognizable biomimetic topology that promotes tactile and visual interaction by a child.

16. The biomimetic sensory engagement system of claim 15, wherein the recognizable biomimetic form corresponds to a dolphin configuration in which the prehensile tail forms a dorsal hook.

17. The biomimetic sensory engagement system of claim 15, wherein the recognizable biomimetic form corresponds to a turtle configuration having fin-like lateral protrusions defining auxiliary chew zones.

18. The biomimetic sensory engagement system of claim 15, wherein the tactile-texture zones mimic anatomical surface variations of the represented animal.

19. The biomimetic sensory engagement system of claim 15, further comprising a handle loop integrally formed with the body and dimensioned for baby grasping.

20. A method of using the biomimetic sensory engagement system of claim 15, the method comprising the steps of:
providing the system to a child;
allowing the child to chew the pacifier-shaped oral-engagement portion corresponding to the head region of the represented animal; and
compressing the hollow torso portion to emit an audible sound through any included squeaker element, thereby reinforcing sensory feedback and recognition of the biomimetic form.
